(19) United States
(12) Patent Application Publication
(10) Pub. No.: US 2025/0302917 A1
(43) Pub. Date: Oct. 2, 2025

(54) METHODS OF REDUCING THE SIGNS OR SYMPTOMS OF DERMAL DISORDERS WITH TOPICALLY APPLIED SILK FIBROIN

(71) Applicant: Cocoon Biotech Inc., Mansfield, MA (US)

(72) Inventors: Ailis Tweed-Kent, Mansfield, MA (US); Michael Santos, Mansfield, MA (US); Scott Delisle, Mansfield, MA (US); Peter Mitchell, Mansfield, MA (US)

(21) Appl. No.: 18/862,286

(22) PCT Filed: Apr. 28, 2023
(86) PCT No.: PCT/US2023/020349

§ 371 Date: Nov. 1, 2024

Related U.S. Application Data
(60) Provisional application No. 63/337,325, filed on May 2, 2022.

(51) Int. Cl. A61K 38/17; A61P 17/00; A61P 17/10

(52) U.S. Cl. CPC A61K 38/1767; A61P 17/00; A61P 17/10

(57) ABSTRACT

Described herein is a method of reducing a sign or symptom of a dermal disorder in a subject in need of treatment for the dermal disorder by topically applying a topical silk fibroin composition once or more daily to the site of the dermal disorder, wherein the silk fibroin formulation includes no additional dermal therapeutic agents. Also included are methods of reducing topical steroid use in a subject in need of treatment for a dermal disorder.

### CROSS-REFERENCE TO RELATED APPLICATIONS

This application claims priority to U.S. Provisional Application 63/337,325 filed on May 2, 2022, which is incorporated herein by reference in its entirety.

### FIELD OF THE DISCLOSURE

The present disclosure is related to methods of topically applying silk fibroin to subjects in need thereof.

### BACKGROUND

Silk fibroin, and in particular silk fibroin hydrogels have been proposed as delivery vehicles for a wide range of active agents, including non-steroidal anti-inflammatory drugs as described in U.S. 2019/00038756. U.S. 2018/00008522 describes silk fibroin compositions including a therapeutic agent such as a therapeutic agent for the treatment of psoriasis. Silk fibroin itself is not disclosed as having any therapeutic activity, but rather as a formulation component for delivery of therapeutic agents.

Described herein are therapeutic uses of fibroin for the treatment of dermal diseases.

### BRIEF SUMMARY

In an aspect, a method of reducing a sign or symptom of a dermal disorder in a subject in need of treatment for the dermal disorder comprises topically applying a topical silk fibroin composition once or more daily to the site of the dermal disorder, wherein the silk fibroin formulation comprises no additional dermal therapeutic agents.

In another aspect, a method of reducing topical steroid use in in a subject in need of treatment for a dermal disorder comprises topically applying a topical steroid formulation once or more daily to a site of the dermal disorder, topically applying a topical silk fibroin composition once or more daily to the site of the dermal disorder, and subsequent to topically applying the topical silk fibroin composition, topically applying the topical steroid formulation less than the once or more daily, or discontinuing the topical steroid formulation, while continuing topically applying the topical silk fibroin composition once or more daily, wherein the silk fibroin formulation comprises no additional dermal therapeutic agents.

### BRIEF DESCRIPTION OF THE DRAWINGS

FIG. 1 shows the improvement in keratolysis exfoliativa upon topical application of a silk fibroin solution. Top photo is pre-treatment, bottom photo is post-treatment.

FIG. 2 shows the improvement in facial redness/acne pre treatment (on left) and post treatment (on right)

FIG. 3 shows the improvement in psoriasis upon topical application of a silk fibroin solution.

The above-described and other features will be appreciated and understood by those skilled in the art from the following detailed description, drawings, and appended claims.

### DETAILED DESCRIPTION

Described herein are methods of reducing the signs or symptoms of dermal disorders and/or reducing the use of topical steroids in patients suffering from dermal disorders by topically applying silk fibroin formulations to the site of the symptom of the dermal disorder. It was unexpectedly found herein that topical application of silk fibroin, in the absence of an additional dermal therapeutically active agent, reduced symptoms of dermal diseases and reduced or eliminated the use of topical steroids.

In an aspect, a method of reducing a sign or symptom of a dermal disorder in a subject in need of treatment for the dermal disorder comprises or consists of topically applying a topical silk fibroin composition once or more daily to the site of the dermal disorder, wherein the silk fibroin formulation comprises no additional dermal therapeutic agents. In an aspect, silk fibroin is the only topically applied agent for the treatment of the dermal disorder.

In another aspect, a method of reducing topical steroid use in in a subject in need of treatment for a dermal disorder comprises topically applying a topical steroid formulation once or more daily to a site of the dermal disorder, topically applying a topical silk fibroin composition once or more daily to the site of the dermal disorder, and subsequent to topically applying the topical silk fibroin composition, applying the topical steroid formulation less than the once or more daily, or discontinuing the topical steroid formulation, while continuing topically applying the topical silk fibroin composition once or more daily, wherein the silk fibroin formulation comprises no additional dermal therapeutic agents.

For example, if the topical steroid is being administered once daily, it may be administered once every other day or less frequently subsequent to topically applying the topical silk fibroin composition. If the topical steroid is being applied twice daily, it may be administered once daily subsequent to topically applying the topical silk fibroin composition. If the topical steroid is being administered 2-3 times daily, it may be administered once daily subsequent to topically applying the topical silk fibroin composition.

In an aspect, the dermal disorder comprises eczema, psoriasis, keratolysis exfoliativa, facial redness, atopic dermatitis, contact dermatitis, seborrheic dermatitis, acute radiation dermatitis, rosacea, vitiligo, actinic keratosis, seborrheic keratosis, urticaria, herpes zoster, acne vulgaris, warts, molluscum contagiosum, spider angioma, cherry angioma, scabies, Tinea corporis, Tinea capitis, Tinea pedis, Tinea cruris, Tinea versicolor, Pityriasis rosea, Erythema nodosum, Erythema multiforme, Lichen planus, Lichen simplex chronicus, Lichen sclerosus, discoid lupus erythematosus, pemphigous vulgaris, bullous pemphigoid, stevens-johnson syndrome, or toxic epidermal necrolysis, and the like.

The terms eczema and atopic dermatitis are often used interchangeably and are inflammatory skin conditions. Eczema is a skin condition that makes skin itchy and red and can also be characterized by dry skin.

Psoriasis is chronic inflammatory skin disease often characterized by red, itchy, scaly patches on the knees, elbows, scalp and trunk, for example.

Facial redness can be caused by different dermal disorders or can be facial redness of unknown origin.

Contact dermatitis is s a red, itchy rash caused by direct contact with a substance or an allergic reaction to it. Soaps, cosmetics, plants and fragrances can cause contact dermatitis.

Seborrheic dermatitis affects the scalp, causing scaly patches, red skin, and persistent dandruff.

Acute radiation dermatitis typically occurs within 90 days of exposure to radiation and can be evidenced by reddening of the skin, peeling skin, or even necrosis of skin.

Rosacea is a skin condition characterized by blushing or flushing and/or visible blood vessels in the face. It may also be characterized by small bumps.

Vitiligo is a condition in which the skin loses pigment cells.

Keratolysis exfoliative is a common skin condition in which there is focal peeling of the palms and less often the soles of the feet.

Exemplary symptoms of a dermal disorder include redness, itching, pecling, scaling, flaking, thin skin, dry skin, flushing, blushing, depigmentation, roughness, swelling/edema, warmth, inflammation, and the like. Symptoms are disruptions in normal function caused by a disease or disorder.

Exemplary signs include erythema, peeling, scaling, flaking, depigmentation, hyperpigmentation, pruritus, hives, urticaria, thickened skin, tight skin, weeping, discharge, purulence, lesions (i.e., raised, non-raised, blanching, non-blanching, colored, erythematous, linear, clustered, annular, arciform, geographic, serpiginous), pustules, patches, papules, macules, fissures, vesicles, purpura, dermatomal lesions, nevi, ulcers, nodules, comedones, cysts, scars, and the like. Signs are objective evidence of a disease or disorder in a subject.

Exemplary topical steroids include clobetasone, hydrocortisone, beclomethasone, betamethasone, clobetasol, diflorasone, fluocinolon, fluocinonide, halobetasol, desoximetasone, halcinonide, amcinonide, triamcinolone, mometasone, their pharmaceutically acceptable salts, and the like.

Silk fibroin can be prepared by methods known in the art and/or the methods described herein. In some embodiments, silk fibroin may be obtained from the silkworm species Bombyx mori. Other examples of silk producer species include, but are not limited to, Bombyx mandarina, Bombyx sinesis, Anaphe moloneyi, Anaphe panda, Anaphe reticulate, Anaphe ambrizia, Anaphe carteri, Anaphe venata, Anapha infracta, Antheraea assamensis, Antheraea assama, Antheraea mylitta, Antheraea pernyi, Antheraea yamamai, Antheraea polyphemus, Antheraea oculea, Anisota senatoria, Apis mellifera, Araneus diadematus, Araneus cavaticus, Automeris io, Atticus atlas, Copaxa multifenestrata, Coscinocera hercules, Callosamia promethea, Eupackardia calleta, Eurprosthenops australis, Gonometa postica, Gonometa rufobrunnea, Hyalophora cecropia, Hyalophora euryalus, Hyalophora gloveri, Miranda auretia, Nephila madagascarensis, Nephila clavipes, Pachypasa otus, Pachypasa atus, Philosamia ricini, Pinna squamosa, Rothschildia hesperis, Rothschildia lebeau, Samia Cynthia, and Samia ricini.

In an aspect, fibroin is produced by providing raw silk (e.g., unpurified silk such as silk yarn), the raw silk comprising fibers containing silk fibroin and sericin. First, the raw silk is degummed in a salt solution, specifically a sodium carbonate solution with a sodium carbonate concentration of 0.02 to 0.5 M sodium carbonate at a temperature of about 60° C. to about 100° C., and for a time of greater than 60 minutes to about 480 minutes. In a preferred aspect, degumming is performed in 0.5 M sodium carbonate at 85° C. for 240 to 360 minutes. In an aspect, degumming provides degummed silk fibers having a sericin concentration of 0-0.5 wt %.

After the degumming, the silk fibroin fibers are further processed by dissolving, preferably in aqueous solution. Dissolving preferably includes using 5M to 13M lithium bromide for 1 hour to overnight at 50° C. to 100° C. to provide dissolved silk fibers, or dissolving the degummed silk fibers using a mixture of calcium chloride, ethanol, and water in a molar ratio of 1:2:8 for 1 to 2 hours to overnight at 50° C. to 100° C., specifically 80° C., to provide dissolved silk fibers. In a specific aspect, 10 wt % to 20 wt % silk fibroin is dissolved in 9.3M lithium bromide at 60° C. for 16 hours (overnight).

In a preferred embodiment, the degummed silk fibroin is dissolved in 5 to 13 M LiBr. The concentration of LiBr may be 9.3 M. Dissolving in LiBr can be done at 60-80° C. for 16 hours (overnight).

After the silk fibroin fibers are dissolved, they can be diluted prior to further purification. In an aspect, the dissolved silk fibers are diluted in water to provide a concentration of 5 to 20% w/v silk fibroin fibers. Optionally the diluted fibroin solution is filtered through a polypropylene, polyethersulfone, nylon, or cellulose, diatomaceous earth, perlite depth prefilter to remove particulates and provide a clarified silk fiber solution.

The diluted silk fibroin fibers can be purified using dialysis or tangential flow filtration (TFF) using a regenerated cellulose or polyethersulfone filter, and concentrating and recovering processed silk fibroin from the TFF. For example, the silk solution is concentrated 2×, and then diafiltered to remove the chaotropic agents, i.e., lithium bromide/calcium chloride and ethanol. Diafiltration can be performed with a silk fibroin solution retentate at pH 3.0-11.0 with a replacement feed of water, water at pH 3.0-11.0, salt solution, i.e., sodium chloride, potassium chloride, (10 mM-500 mM) at pH 3.0-11.0, buffer, i.e., sodium phosphate, potassium phosphate, tromethamine, at 10 mM-250 mM, at pH 3.0-11.0, buffer containing 10 mM-500 mM salt pH 3.0-11.0, or a combination thereof.

The replacement feed can be water. Diafiltration is performed for 5-15 diavolumes, or until a sufficient amount of the chaotropic agent is removed.

The molecular weight of the silk fibroin is not critical, and can be, for example, from 1 kDa to 100 kDa, but preferably less than 80 kDa. In an aspect, the molecular weight is about 30 to about 50 kDa, specifically 30-42 kDa. Molecular weight is preferably determined using size exclusion chromatography (SEC).

In specific aspects, the topical formulations are in the form of solutions, gels, ointments, creams, lotions, serums, and the like.

An exemplary solution includes silk fibroin dissolved in water, glycerol, propylene glycol, and isopropyl alcohol.

Exemplary gels include fibroin suspended in a base of water and a thickening agent, such as xanthan gum. Gels in which the dispersed liquid molecules include water are referred to herein as “hydrogels.” Gels tend to be lighter and less moisturizing than creams or lotions, making them a suitable option for certain types of skin, such as oily or acne-prone skin. Gels can be cooling, refreshing and more readily absorbed than many other topical formulations.

In some embodiments, formulations include creams or moisturizers. As used herein, the term cream refers to a thick mixture of various ingredients. Creams may be semisolid dosage forms containing more than 20% water or volatile components. Creams may have four main ingredients: water, oil, emulsifier, and a thickening agent, in addition to silk fibroin.

Lotions are low-viscosity topical preparations intended for application to the skin intended to simply moisturize the skin or to treat or prevent skin diseases and irritations. Compared to other topical and spreadable formats, lotions may have a higher water content. Exemplary lotions are oil-in-water emulsions using a substance such as cetearyl alcohol to keep the emulsion together, but water-in-oil lotions may also be formulated. A skin care lotion, cream or gel emulsion (that is mixtures of oil and water) may include aqueous and oily phases, an emulgent to prevent separation of these two phases. A wide variety of other ingredients such as fragrances, glycerol, petroleum jelly, dyes, preservatives, proteins, and stabilizing agents are commonly added to lotions. An exemplary lotion in the form of an emulsion includes an aqueous phase including 1-3 wt % fibroin, and an oil phase including MCT, coconut oil and avocado oil. Another exemplary lotion includes an oil phase including MCT oil, shea butter, and cocoa butter and an aqueous phase consisting of 3% (w/v) SF in water. Lotions using silk fibroin as an emulsifier and thickener were prepared using jojoba oil as the primary emollient.

The formulations may include solvents. Exemplary solvents for dissolving water-soluble ingredients include glycerin, propanediol 1,3 and propylene glycol.

The formulations may also include pH adjusters. pH adjusters are ingredients used to adjust the pH (acidity or alkalinity) of a finished product. The optimum pH for most products is between 4.5 and 7.

In some embodiments, the formulations may include thickeners. Thickeners are used to enhance the consistency, volume and viscosity of cosmetic products, thereby providing more stability and better performance. While some thickeners also have emulsifying or gelling properties, many thickeners have the ability to retain water on the skin and act therefore as moisturizers. Thickeners can be completely natural like waxes but also synthetic or semi-synthetic. They are derived from various sources and consist of very different molecular structures including polysaccharides, proteins, polymers, alcohols, silicones or waxes. Waxes are complex mixtures of alcohols, fatty acids and esters. They are harder, less greasy and more brittle than fats, and are very resistant to moisture, oxidation and microbial degradation.

Exemplary concentrations of Silk fibroin for the reduction of a symptom of a dermal disorder and/or reducing topical steroid use are 0.1 to 5.0 wt %. It is preferred, but not necessary, that the fibroin have a molecular weight of ≤80 kDa as measured by SEC. The invention is further illustrated by the following non-limiting examples.

### EXAMPLES

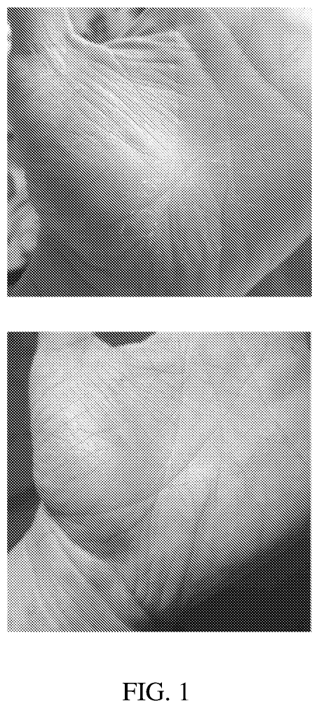

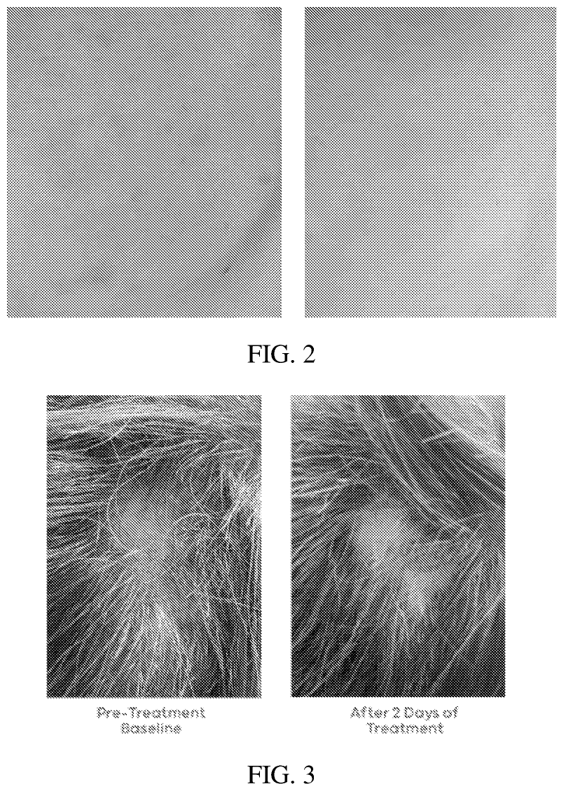

As shown in FIGS. 1-3, a 2.0 wt % silk fibroin solution or serum applied topically twice daily reduced a symptom and/or sign of keratolysis exfoliative, facial redness and acne, and psoriasis.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. A method of reducing a sign or symptom of a dermal disorder in a subject in need of treatment for the dermal disorder, comprising
topically applying a topical silk fibroin composition once or more daily to the site of the dermal disorder, wherein the silk fibroin formulation comprises no additional dermal therapeutic agents,
wherein the dermal disorder is eczema, psoriasis, keratolysis exfoliativa, facial redness, or atopic dermatitis,
wherein the sign or symptom is not skin dryness.

2. The method of claim 1, wherein the dermal disorder is eczema or psoriasis.

3. The method of claim 1, wherein the sign is erythema, peeling, scaling, flaking, depigmentation, hyperpigmentation, pruritus, hives, urticaria, thickened skin, tight skin, weeping, discharge, purulence, lesions, pustules, patches, papules, macules, fissures, vesicles, purpura, dermatomal lesions, nevi, ulcers, nodules, comedones, cysts, scars, or a combination thereof.

4. The method of claim 1, wherein the symptom is redness, itching, peeling, scaling, flaking, thin skin, flushing, blushing, depigmentation, roughness, swelling/edema, warmth, inflammation, or a combination thereof.

5. The method of claim 1, wherein the silk fibroin formulation is in the form of a solution, a gel, an ointment, a cream, a lotion, or a serum.

6. The method of claim 1, wherein the silk fibroin formulation has a silk fibroin concentration of 0.1 to 5.0 wt %.

7. The method of claim 1, wherein the silk fibroin has a molecular weight of ≤80 kDa.

8. The method of claim 1, wherein the silk fibroin is the only topically applied agent for the treatment of the dermal disorder.

9. A method of reducing topical steroid use in in a subject in need of treatment for a dermal disorder, comprising
topically applying a topical steroid formulation once or more daily to a site of the dermal disorder,
topically applying a topical silk fibroin composition once or more daily to the site of the dermal disorder, and
subsequent to topically applying the topical silk fibroin composition, applying the topical steroid formulation less than the once or more daily, or discontinuing the topical steroid formulation, while continuing topically applying the topical silk fibroin composition once or more daily,
wherein the silk fibroin formulation comprises no additional dermal therapeutic agents,
wherein the dermal disorder is eczema, psoriasis, keratolysis exfoliativa, atopic dermatitis,
wherein the subject is not suffering from skin dryness.

10. The method of claim 9, wherein the dermal disorder is eczema or psoriasis.

11. The method of claim 9, wherein the topical steroid comprises clobetasone, hydrocortisone, beclomethasone, betamethasone, clobetasol, diflorasone, fluocinolon, fluocinonide, halobetasol, desoximetasone, halcinonide, amcinonide, triamcinolone, mometasone, or a pharmaceutically acceptable salt thereof.

12. The method of claim 9, wherein the silk fibroin formulation is in the form of a solution, a gel, an ointment, a cream, a lotion, or a serum.

13. The method of claim 9, wherein the silk fibroin formulation has a silk fibroin concentration of 0.1 to 5.0 wt %.

14. The method of claim 9, wherein the silk fibroin has a molecular weight of ≤80 kDa.
